(19) United States
(12) Patent Application Publication
(10) Pub. No.: US 2009/0074751 A1
(43) Pub. Date: Mar. 19, 2009

(54) GROWTH FACTOR FRACTION COMPOSITIONS AND METHODS

(75) Inventor: Joseph C. Ramaekers, Aptos, CA (US)

Correspondence Address: FOX ROTHSCHILD LLP;PRINCETON PIKE CORPORATE CENTER, 2000 Market Street, Tenth Floor, Philadelphia, PA, 19103, US

(73) Assignee: RAMAEKERS NUTRITION, INC., Santa Cruz, CA (US)

(21) Appl. No.: 12/233,231

(22) Filed: Sep. 18, 2008

Related U.S. Application Data
(60) Provisional application No. 60/973,440, filed on Sep. 18, 2007.

(51) Int. Cl. A61K 39/395; A61K 38/02; A61K 36/00; A61K 33/42; A61K 33/14; A61K 33/06; A61P 3/00

(52) U.S. Cl. 424/130.1; 514/2; 424/725; 424/601; 424/663; 424/682

(57) ABSTRACT

Compositions and formulations are provided comprising a growth factor fraction and at least one glucan. Also provided are methods for making the compositions and formulations. Methods of promoting growth in an animal using the compositions and/or formulations are also provided.

### FIELD OF THE INVENTION

This invention relates generally to compositions and formulations comprising a growth factor fraction and at least one glucan. Such formulations are useful in providing health benefits to animals, including promoting growth.

### BACKGROUND OF THE INVENTION

The present invention relates to compositions and formulations comprising a growth factor fraction, as well as methods of making the same and methods of administration using the same. Other U.S. patents and U.S. patent applications relate to the present invention, including without limitation, U.S. Pat. Nos. 6,506,413 and 6,962,718, U.S. Patent Provisional Application Nos. 60/573,113, 60/649,363, 60/701,860, and 60/814,777, U.S. patent application Ser. No. 11/762,727, U.S. Patent Application Publication Nos. 2006/0029585 A1, 2006/0073197 A1, and 2007/0128253 A1, all of which are incorporated herein by reference. Also related are PCT publications WO/2002/087599 and WO/2005/112891, incorporated herein by reference.

### BRIEF SUMMARY OF THE INVENTION

In certain aspects, the invention relates to compositions comprising a growth factor fraction. Preferably, the growth factor fraction is combined with at least one glucan. In certain embodiments, the growth factor fraction may be combined with an antibody. In certain aspects, the antibody is contained in an antibody fraction. In certain embodiments, the growth factor fraction and/or the antibody or antibody fraction with which it is combined may be lyophilized.

In further aspects, compositions and formulations of the invention may further comprise additional components. In certain embodiments, the compositions and formulations comprising a growth factor fraction may additionally comprise transfer factor. In certain embodiments, the transfer factor may be lyophilized.

Additional aspects of the invention relate to compositions and formulations comprising a growth factor fraction that is encapsulated by a hydrophobic or lipid coating. Additional components of the compositions may also be encapsulated, including, but not limited to, encapsulated glucans, and encapsulated antibody or antibody fraction, encapsulated transfer factor, and combinations thereof.

Additional aspects of the present invention are directed to methods of making compositions and formulations comprising a growth factor fraction.

Further aspects of the present invention are directed to methods of promoting growth in an animal by administering an effective amount of a composition and/or formulation comprising a growth factor fraction.

Both the foregoing general description and the following detailed description are exemplary and explanatory, and are intended to provide further explanation of the invention as claimed. Other objects, advantages, and novel features will be readily apparent to those skilled in the art from the following detailed description of the invention.

### DETAILED DESCRIPTION OF THE INVENTION

In certain embodiments, the present invention is directed to compositions and formulations comprising a growth factor fraction. In preferred embodiments, the growth factor fraction may be obtained from colostrum. Preferably, compositions comprising growth factor fraction further comprise at least one glucan.

In certain embodiments, the growth factor fraction is combined with an antibody. In particular embodiments, the antibody is contained within an antibody fraction.

In certain embodiments, growth factor fraction may be combined with transfer factor. In certain embodiments, the transfer factor may be obtained from an avian and/or a mammalian source.

Other aspects of the invention are directed to formulations further comprising components, including, but not limited to, nutraceutical ingredients, in addition to the growth factor fraction. In other aspects of the invention, there are provided formulations comprising additional components in addition to growth factor fraction and glucan.

In certain embodiments of the invention, a fraction containing a growth factor or growth factors may be obtained from colostrum. In a preferred embodiment, this growth factor fraction is obtained from bovine colostrum. The colostrum may be fractionated; the fraction comprising material having a molecular weight of approximately 10,000 daltons (Da) and above is designated as the growth factor fraction. The growth factor fraction may include high molecular weight proteins. The fraction obtained that is approximately 10,000 to approximately 150,000 Da is designated the antibody fraction, also known as the antibody-colostrum fraction. The fraction comprising material having a molecular weight of approximately 10,000 Da and below is designated as transfer factor.

In other embodiments, the transfer factor and/or antibody is obtained from an avian source. One such source is chickens that are inoculated with or exposed to one or more pathogens. Inoculation with pathogens may be accomplished by any effective means. In one embodiment, chickens are given a feed mixture containing excrement from an animal, including without limitation, a human, a fish, a goat, a llama, an alpaca, a pig, a sheep, a cow, and a horse. The excrement will contain a large variety of pathogens and upon administration of a feed to an animal, it will develop transfer factor and/or antibodies to such pathogens. Avian transfer factor and avian antibodies can then be obtained from the eggs produced by the above-treated chickens. In certain embodiments of the invention, avian transfer factor and avian antibodies may be found in whole egg yolks. As a non-limiting example, the antibodies of avian source are supplied as powdered whole egg yolks (which is believed to also contain transfer factor).

Additionally, transfer factor and antibodies may be derived from any suitable source, as described, for example, in U.S. Pat. Nos. 4,816,563; 5,080,895; 5,840,700; 5,883,224; and 6,468,534; and U.S. patent application Ser. No. 11/762,727, the contents of which are hereby incorporated by reference herein.

Lyophilization

The present invention also provides compositions and formulations that have one or more lyophilized component(s). Lyophilization or “freeze-drying” is a process well known to those of ordinary skill in the art. For example, some techniques of lyophilization are described in Akers, Michael J., Chapter 41 in Remington The Science and Practice of Pharmacy, 828 (David B. Troy ed., Lippincott Williams & Wilkins 2006), which is incorporated herein by reference. In certain embodiments, formulations and/or compositions of the present invention may include lyophilized growth factor fraction. In certain embodiments, growth factor fraction, which may be lyophilized, may be combined with antibody or an antibody fraction, which may, in certain embodiments, be lyophilized. In other embodiments, transfer factor, which may be combined with growth factor fraction, may be lyophilized. In other embodiments, additional components of formulations and compositions of the invention may be lyophilized, including, without limitation, other peptides and proteins.

### Formulations

In certain embodiments of the invention, growth factor fraction is provided in a formulation that further comprises one or more additional ingredients. In some embodiments, growth factor fraction and at least one glucan is provided in a formulation that further comprises one or more additional ingredients. In certain embodiments, the growth factor fraction may be lyophilized. In certain embodiments, the optional antibody or antibody fraction may be lyophilized.

In a preferred embodiment, growth factor fraction is present in the formulation in the amount of about 10 mg to about 12 gm/oz, more preferably about 100 mg to about 6 gm/oz and most preferably about 10 mg to about 3 gm/oz. In certain preferred embodiments, such a formulation comprising transfer factor is provided to an animal in an amount of about 1 oz per 1000 lb of animal.

In certain embodiments of the invention, formulations are provided which comprise glucans. Glucans may be derived from any suitable source, including, but not limited to, fungi, oats, and yeast. Preferably, glucans are present in or derived from fungi. In certain embodiments, the glucans which may be included in the formulations are present in whole fungi. In certain preferred embodiments, glucans are present in or derived from Cordyceps, more preferably, Cordyceps sinensis.

In certain embodiments, glucans are derived from hybrid strains of fungi. In a preferred embodiment the hybrid glucans used in the invention are present in, or derived from, hybrid strains of Cordyceps and in particular Cordyceps sinensis. One technique to induce the hybridization of Cordyceps involves plating two different strains or species on a single agar plate which has been inoculated with rattlesnake venom as described in, for example, U.S. Patent Application Publication No. 2006/0073197, published Apr. 6, 2006, and U.S. Patent Application Publication No. 2007/0128253, published Jun. 7, 2007, each of which is incorporated herein by reference. In a preferred embodiment, the hybrid strain producing the hybrid glucans that may be used in compositions and formulations of the invention is Cordyceps sinensis Alohaensis, which is available from Pacific Myco Products, Santa Cruz, Calif.

In addition to Cordycep sinensis hybrids, suitable sources of glucans may include, but are not limited to, Agaricus Blazeii, Coriolus Poira Cocos, Inonotus Obliquus, Maitake Mushroom, Shiitake Mushroom, and combinations thereof.

When glucans are used, the formulation preferably contains about 10 mg to about 18 gm of whole organism/oz, more preferably about 100 mg to about 10 gm of whole organism/oz and most preferably about 100 mg to about 5 gm of whole organism/oz.

Equivalent amounts of purified or partially purified glucan as well as the nucleosides associated therewith (e.g., Cordycepin (3′deoxyadenosine), adenosine and N6-(2 hydroxyethyl)-adenosine) may also be used.

In certain embodiments, compositions and formulations comprising growth factor fraction may be combined with minerals, antioxidants, amino acids, and other nutraceuticals.

In certain preferred embodiments of the invention, formulations that may be administered to a subject, may, in addition to growth factor fraction and one or more glucans, include one or more of the following: mammalian antibody-colostrum fraction, avian antibodies or antibody fraction (which may, in certain embodiments, be obtained from whole egg yolk), and transfer factor, which, in preferred form, may be derived from mammalian or avian source.

In certain preferred embodiments, compositions may further comprise one or more of inositol hexaphosphate (Ip6), mannans, olive leaf extract, and phytosterols. In certain preferred embodiments, mannans are derived from Aloe vera. In certain preferred embodiments, phytosterols may be derived from soya bean.

In certain embodiments, compositions may further comprise one or more of lactic acid producing bacteria, ascorbic acid, Vitamin A, Vitamin D3, Vitamin E, Vitamin B1, Vitamin B2, Vitamin B 12, dipotassium phosphate, potassium chloride, magnesium sulfate, and calcium pantothenate.

In certain embodiments, the invention provides compositions and formulations in which one or more components are encapsulated. Encapsulation may be achieved by mixing the component to be encapsulated with a hydrophobic substance or a lipid to form a coating around the component. Encapsulation may protect labile components from inactivation in the gastrointestinal tract. Such encapsulation may be important especially in the case of ruminants where digestion within the rumen has been found to interfere with the administration of certain labile factors. Enhanced bioavailability has been demonstrated, for example, when a transfer factor is encapsulated and administered to ruminants.

Previous use of non-encapsulated transfer factor in ruminants, e.g., cows, produced significant beneficial results. See, e.g. U.S. Patent Publication 2003/0077254, published Apr. 24, 2003 incorporated herein by reference in its entirety. Subsequently, it was discovered that transfer factor was not stable by oral administration in a stressed population of cattle. After discovering that transfer factor is inactivated in vitro in the presence of rumen fluid and flora, it was determined that prior success with transfer factor in ruminants was due to the presence of the esophageal groove. When not stressed, the esophageal groove provides partial bypass of the rumen. However, in a stressed population the esophageal groove closes and shunts the transfer factor formulation into the rumen. It was discovered that encapsulating transfer factor and/or glucans with a hydrophobic substance or a lipid to form an encapsulated formulation is sufficient to provide substantial by-pass of (e.g., 85%) of the rumen even in a stressed population.

While not seeking to be bound by any theory or theories, it is believed that encapsulation of growth factor fraction may increase its bioavailability upon administration to fermenting animals, such as adult ruminants.

In preferred embodiments, the growth factor fraction is encapsulated by mixing with a hydrophobic substance or a lipid to form a coating around the growth factor(s). In additional embodiments, one or more additional components such as antibody, antibody fraction, transfer factor and/or glucans may be encapsulated. Other optional components of compositions and formulations of the invention may be encapsulated, such as, without limitation, inositol hexaphosphate, olive leaf extract, mannans, phytosterol, vitamin C and mixtures thereof. The growth factor fraction, antibody or antibody fraction, and/or transfer factor may each be individually encapsulated or encapsulated as a mixture. Alternatively, the entire formulation can be encapsulated. The encapsulated component(s) and/or formulation can be produced in a variety of ways. In a preferred embodiment, each of the growth factor fraction, glucans, antibody or antibody fraction, and/or transfer factor in the formulation may be encapsulated as described in U.S. Pat. Nos. 5,190,775, 6,013,286 and U.S. Application 2003/0129295, each of which is incorporated herein by reference in its entirety.

In certain embodiments, glucans of the formulation may be encapsulated, preferably with a hydrophobic or lipid coating. It is preferred that the amount of hydrophobic or lipid coating be between about 25% and 150 wt/% of the glucan, about 50-150 wt %, or about 75-125 wt/%, with an equal weight being most preferred.

The formulation described in Example 2 provides an exemplary encapsulated growth factor formulation. In certain embodiments, the growth factor formulation includes encapsulated growth factor fraction and one or more glucans. In certain preferred embodiments, the formulation may further comprise encapsulated antibodies and/or encapsulated transfer factor. It is preferred that the glucan portion of this formulation also be encapsulated. In preferred form, additional components include one or more of the following that has been encapsulated: inositol hexaphosphate (IP6), phytosterol, olive leaf extract, mannans, and Vitamin C. Additional optional components may include one or more of Vitamin A, Vitamin D3, Vitamin E, Vitamin B1, Vitamin B2, Vitamin B 12, dipotassium phosphate, potassium chloride, magnesium sulfate, calcium pantothenate, and lactic acid producing bacteria. In a preferred embodiment, all of the foregoing are included in a growth factor formulation.

The growth factor fraction may be encapsulated with a hydrophobic or lipid coating that is preferably between about 25% and about 150 wt/% of the growth factor fraction, about 50-150 wt/% and about 75-125 wt/%, with an equal weight of growth factor fraction and hydrophobic or lipid coating being most preferred.

A variety of other methods for rumen by-pass are known. In one embodiment, the encapsulated or non-encapsulated formulation is directly injected (subcutaneously, intramuscularly, or intravenously) to by-pass not only the rumen but also the entire digestive system. Similarly, intravaginal, intrarectal or other direct administration to mucus membranes, such as the eye subconjunctival, by-pass the digestive system and the rumen in particular. Alternatively, the formulation can be mixed with various solvents which allow for direct skin absorption. Furthermore, methods are known in the art to stimulate opening of the esophageal groove in various ruminants and such opening allows for immediate passage of an orally administered formulation to the gastrointestinal tract, by-passing the rumen.

In additional embodiments of the invention, additional components may be used in the formulation. For example, IP6, olive leaf extract, aloe extract and/or vitamin C may be used. In preferred embodiments, IP6 is present at between 10 mg and 3 gm/oz, or one preferably between 100 mg and 2 gm/oz, and most preferably between 100 mg and 1 gm/oz. Olive leaf extract is preferably present in the amount of 2 mg to 2 gm/oz, more preferably between 5 mg and 1 gm/oz, and most preferably between 5 mg and 500 gm/oz. Aloe extract is preferably present at between 2 mg and 1000 mg, more preferably between 5 and 500 mg/oz, and most preferably between 5 and 250 mg/oz. Vitamin C may be present at between 10 mg/oz and 10 gm/oz, or preferably between 100 mg and 8 gm/oz, and most preferably between 100 mg and 5 gm/oz.

### Methods of Administration

In certain embodiments, the present invention provides methods involving administration of compositions and/or formulations according to the invention to a subject. As used herein, the “subject” is preferably an animal, including, but not limited to, a calf.

In certain embodiments, the present invention provides methods of administering to a subject a composition or a pharmaceutical formulation, as described herein. Embodiments of the invention are directed to use of compositions and formulations comprising growth factor fraction as described herein for promoting growth in an animal.

In certain embodiments, the growth factor formulations find use in increasing food conversion efficiency. Food conversion efficiency is the rate at which an organism can convert food to body mass, and is also known in the cattle industry as feed conversion efficiency. The compositions and formulations described herein find use in a number of aspects of overall organismal health and development. In certain embodiments, methods of improving feed conversion in an animal subject comprises the administration to the subject of compositions and formulations comprising growth factor fraction.

The growth factor formulations of the present invention include pharmaceutical compositions suitable for administration. In a preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as sodium acetate; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the pharmaceutical compositions may be added in a micellular formulation; see U.S. Pat. No. 5,833,948, hereby expressly incorporated by reference in its entirety.

In one aspect, the components of the compositions and pharmaceutical formulations of the present invention may have an effect upon administration individually, but upon administration in one or more combinations, have an effect that is synergistic. By “synergistic” is meant an enhancement of the effect of one or more combined components in a more than additive fashion relative to the effect of each component when used alone.

Combinations of pharmaceutical compositions may be administered. Moreover, the compositions may be administered in combination with other therapeutics.

The following examples serve to more fully describe the manner of using the above-described invention. It is understood that these examples in no way serve to limit the true scope of this invention, but rather are presented for illustrative purposes.

### EXAMPLE 1

### Effects of a Formulation Containing Growth Factor Fraction on Sale Barn Stressed Calves

The following study was performed on Holstein calves obtained at 1 to 3 days of age. The calves were sale barn stressed; bought from an order buyer who collects calves, usually all bull calves to be castrated, from a large number of dairies. When the order buyer's truck is full, usually holding several hundred head, the animals are taken to a farm such as the one associated with this study. All of the calves used in this study were from one truck load. They arrive at the feeding facility one to three days old, highly stressed. They were placed in hutches individually, the operation having over 500 hutches. The animals were fed initially with milk replacer supplied in a nipple bucket. Since some calves do not take readily to the nipple bucket, the number of days to sucking has been noted. After several days, feed was placed in a trough for the animals. Since animals that start eating full feed there generally have less trouble with disease, time to full feed has been noted. The animals continued feeding from the milk nipple bucket for approximately 60 days. Fresh water was supplied daily in a separate bucket at all times.

After about 60 days the animals were transferred to growing pens on full feed, and weaned off milk altogether. Calves are usually vaccinated, wormed, and castrated in the first five to ten days at the calf growing facility.

Forty-eight (48) calves were divided into three groups. One group of 16 calves (Group 1) was given Formulation 1 comprising growth factor fraction. The formulation was administered by dissolving it in the milk replacer. One ounce of Formulation 1 was administered to the animals on each of Day 1, Day 2 and Day 12.

### Formulation 1

Amounts are given per ounce of Formulation

| | | |
|---|---|---|
| Freeze dried (lyophilized) growth factor fraction | 2000 | mg |
| (Available from Sterling Technology, Inc., | | |
| Brookings, SD) | | |
| Avian antibodies against: | 3000 | mg |
| E. Coli, Rota and corona virus, Salmonella | | |
| (Avian antibodies supplied as whole egg yolk | | |
| from chickens immunized with the indicated | | |
| pathogens. Available from Labelle, Inc., | | |
| Bellingham, WA) | | |
| Poly R-Glucans derived from: | | |
| Cordyceps sinensis hybrid | 1000 | mg |
| Agaricus Blezeii | | |
| Coriolus | | |
| Poira Cocos | | |
| Inontus Obliquus | | |
| Maitake | | |
| Shiitake | | |
| (Available from Aloha Medicinals Inc., | | |
| Carson City, NV) | | |
| Vitamin A | 12150 | IU |
| Vitamin D3 | 3125 | IU |
| Vitamin E | 400 | IU |
| Vitamin B1 | 35 | mg |
| Vitamin B2 | 35 | mg |
| Vitamin B 12 | 4 | mg |
| Dipotassium Phosphate | 4000 | mg |
| Potassium Chloride | 566 | mg |
| Magnesium Sulfate | 226 | mg |
| Calcium Pantothenate | 62 | g |
| Ascorbic Acid | 62 | mg |
| Lactic Acid Bacteria | 2.5 | Billion CFU |

Group 2 consisted of 16 calves to which the following Comparative Formulation was administered by dissolving it in the milk replacer on Day 1, Day 2, and Day 12. Amounts are given per ounce of formulation. This formulation is available from 4Life® Research.

| | | |
|---|---|---|
| Transfer factor | 1450 | mg |
| (Proprietary mixture from avian and | | |
| mammalian sources) | | |
| Proprietary Blend: | 1000 | mg |
| Ip6 (Inositol hexaphosphate) | | |
| Soya bean extract (Phytosterols) | | |
| Olive leaf extract (Olea europea) | | |
| Mannans (from Aloe vera leaf) | | |
| Poly-R ™ Proprietary Blend: | 1000 | mg |
| Cordyceps sinensis hybrid | | |
| Agaricus Blazeii Extract | | |
| Coriolus | | |
| Poira Cocos | | |
| Inonotus Obliquus | | |
| Maitake Mushroom | | |
| Shiitake Mushroom | | |
| Vitamin A | 12,150 | IU |
| Vitamin D3 | 3125 | IU |
| Vitamin E | 400 | IU |
| Vitamin B1 | 35 | mg |
| Vitamin B2 | 35 | mg |
| Vitamin B12 | 4 | mg |
| Dipotassium Phosphate | 4000 | mg |
| Potassium Chloride | 566 | mg |
| Magnesium Sulfate | 226 | mg |
| Calcium Pantothenate | 62 | mg |
| Ascorbic Acid | 62 | mg |
| Lactic Acid Bacteria | 2.8 | Billion CFU |

Group 3 (Controls) consisted of 16 calves that were not administered either of the above formulations.

### Results

For Group 1 consisting of 16 baby Holstein calves that received one ounce of Formulation 1 on days 1-2-12:
two animals required treatments with 1500 IU of Penicillin each; both responded to the treatment
Mortality=0
animals were purchased in August of 2005, sold in January of 2007 (after 403 days)
Finish weight—animals sold at a weight of 1340 to 1450 pounds; average finish weight approximately 1420 pounds
All sold at USDA Grade Choice
   Behavior All calves sucking by day 3 and on full feed by day 7.
   Condition: alert, active clearly good flesh gaining weight.
   Group 2 consisting of 16 Holstein calves bought at the same time, which received the Comparative Formulation:
required treatment of 5 calves with 1500 IU of Penicillin; all responded to this treatment
Mortality=0
animals purchased in August of 2005, sold March of 2007, (time to finish was 463 days)
animals sold at weight of approximately 1405-1470 pounds, average finish weight of approximately 1430 pounds.
All sold at USDA Grade Choice
   Behavior All calves sucking by day 7 and on full feed by day 14.
   Condition: good flesh gaining weight.
   Group 3 (controls) consisting of 16 Holstein calves purchased at the same time as Groups 1 and 2, to which neither of the above formulations was administered.
All calves in this group were treated over three times each with Nuflor, A180, Banamine, and Micotil. A total of 52 treatments was required.
Mortality=0
Sold in May at approximately 1290 to 1370 pounds, average finish weight of approximately 1320 pounds.
Time to sale (finish) was 484 days
All sold at USDA Grade Select (lower grade than Choice)
   Behavior Calves all sucking on day 10 to 14, still not on full feed by day 24.
   Not as alert, or playful as the other groups.
   Condition Showed poor body condition, including peaked backs and pelvis

The above example demonstrates the beneficial effects, including those relating to growth and to reduced morbidity, achieved in calves administered a formulation comprising a growth factor fraction.

### EXAMPLE 2

Below is described an exemplary formulation, in which certain components are encapsulated, that may prove beneficial for mature ruminants; including, for example, calves older than about 45 to 60 days (the age by which rumination generally begins).

| | | |
|---|---|---|
| Freeze dried (lyophilized) growth factor | 4000 | mg |
| fraction (encapsulated) | | |
| Avian antibodies (encapsulated) | 480 | mg |
| Transfer factor (encapsulated) | 1120 | mg |
| Poly R (encapsulated) | 2000 | mg |
| Proprietary blend (encapsulated) | 300 | mg |
| Phytosterol | | |
| olive leaf | | |
| mannans | | |
| Ip6 | | |
| Vitamin C (encapsulated) | 2000 | mg |
| Vitamin A | 12150 | IU |
| Vitamin D3 | 3125 | IU |
| Vitamin E | 400 | IU |
| Vitamin B1 | 35 | mg |
| Vitamin B2 | 35 | mg |
| Vitamin B 12 | 4 | mg |
| Dipotassium Phosphate | 4000 | mg |
| Potassium Chloride | 566 | mg |
| Magnesium Sulfate | 226 | mg |
| Calcium Pantothenate | 62 | g |
| Lactic Acid Bacteria | 2.5 | Billion CFU |

It will be apparent to those skilled in the art that various modifications and variations can be made in the methods and compositions of the present inventions without departing from the spirit or scope of the invention. Thus, it is intended that the present invention cover the modification and variations of the inventions provided they come within the scope of the appended claims and their equivalents.

The terms and expressions which have been employed are used as terms of descriptions and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope on this invention.

In addition, where features or aspects of the invention are described in terms of Markush group or other grouping of alternatives, those skilled in the art will recognized that the invention is also thereby described in terms of any individual member or subgroup of members of the Markush group or other group.

Unless indicated to the contrary, all numerical ranges described herein include all combinations and subcombinations of ranges and specific integers encompassed therein. Such ranges are also within the scope of the described invention.

The disclosures of each patent, patent application and publication cited or described in this document are hereby incorporated herein by reference, in their entirety.

What is claimed is:

1. A composition comprising a growth factor fraction and at least one glucan.

2. The composition of claim 1 wherein the growth factor fraction is lyophilized.

3. The composition of claim 1 further comprising an antibody.

4. The composition of claim 3 wherein the antibody is contained in an antibody fraction.

5. The composition of claim 3 wherein the antibody is lyophilized.

6. The composition of claim 1 further comprising a transfer factor.

7. The composition of claim 6 wherein the transfer factor is lyophilized.

8. The composition of claim 1 wherein the growth factor fraction is encapsulated by a hydrophobic or lipid coating.

9. The composition of claim 1 wherein the glucan is encapsulated by a hydrophobic or lipid coating.

10. The composition of claim 3 wherein the antibody is encapsulated by a hydrophobic or lipid coating.

11. Composition of claim 1 wherein the composition is encapsulated by a hydrophobic or lipid coating.

12. The composition of claim 1 wherein the growth factor fraction is obtained from colostrum.

13. The composition of claim 3 wherein the antibody is derived from an avian or a mammalian source.

14. The composition of claim 6 wherein the transfer factor is derived from an avian or mammalian source.

15. The composition of claim 1 wherein the glucan is derived from a member selected from the group consisting of Cordyceps sinensis, Agaricus Blazeii, Coriolus Poira Cocos, Inontus obliquus, Maitake, Shiitake, and mixtures thereof.

16. The composition of claim 16 wherein the Cordyceps sinensis is a hybrid.

17. The composition of claim 1 further comprising a member selected from the group consisting of inositol hexaphosphate, olive leaf extract, mannans, and phytosterols.

18. The composition of claim 17 wherein the mannans are obtained from Aloe vera leaf.

19. The composition of claim 1 further comprising a member selected from the group consisting of dipotassium phosphate, potassium chloride, magnesium sulfate, calcium pantothenate, vitamin E, vitamin C, vitamin A, vitamin D3, vitamin B1, vitamin B2, vitamin B 12, and mixtures thereof.

20. A method of making a composition for promoting animal growth comprising the steps of:
A) Fractionating colostrum to obtain a fraction having a molecular weight of about 10,000 Daltons or greater; and
B) Mixing the fraction with at least one glucan.

21. A method of making a formulation for promoting animal growth comprising the steps of:
A) Fractionating colostrum to obtain a fraction having a molecular weight of about 10,000 Daltons or greater;
B) Mixing the fraction with at least one glucan to form a composition; and
C) Combining the composition with a member selected from the group consisting of dipotassium phosphate, potassium chloride, magnesium sulfate, calcium pantothenate, vitamin E, vitamin C, vitamin A, vitamin D3, vitamin B1, vitamin B2, vitamin B 12, and mixtures thereof.

22. A method of promoting growth in an animal comprising administering to the animal a composition comprising a growth factor fraction and at least one glucan.

23. The method of claim 22 wherein the animal is a calf.

24. A method of increasing feed conversion in an animal comprising the step of administering to the animal a composition comprising a growth factor fraction and at least one glucan.

25. The method of claim 24 wherein the animal is a calf.
